(19) United States
(12) Patent Application Publication
(10) Pub. No.: US 2020/0330754 A1
(43) Pub. Date: Oct. 22, 2020

(54) SKIN CARE DEVICE

(71) Applicant: LG ELECTRONICS INC., Seoul (KR)

(72) Inventors: Heejung KIM, Seoul (KR); Munseong KANG, Seoul (KR); Sangwon KIM, Seoul (KR); Yongju YANG, Seoul (KR); Gueisam LIM, Seoul (KR); Sungho HONG, Seoul (KR); Dongwon KIM, Seoul (KR); Heejin PARK, Seoul (KR)

(73) Assignee: LG ELECTRONICS INC., Seoul (KR)

(21) Appl. No.: 16/763,211

(22) PCT Filed: Sep. 3, 2018
(86) PCT No.: PCT/KR2018/010203

§ 371 Date: May 11, 2020

(30) Foreign Application Priority Data
Nov. 15, 2017 (KR) 10-2017-0152037

(51) Int. Cl. A61N 1/32; A61N 1/04; A61N 1/36; A61N 5/06; A45D 44/22

(52) U.S. Cl. CPC A61N 1/328; A61N 1/0416; A61N 1/3603; A61N 2005/0632; A61N 5/062; A45D 44/22; A61N 2005/0626; A61N 5/0616

(57) ABSTRACT

The present invention relates to a skin care device capable of simultaneously performing optical care and fine current care without the occurrence of electrical interference therebetween, and provides a skin care device comprising: a flexible circuit board; at least one light source element provided on one surface of the flexible circuit board; a first case on which the flexible circuit board is mounted; a hole provided in the first case and exposing the light source element; a connection part provided on a surface including the hole of the first case; an electrode patch, which can be attached/detached to/from the connection part, is electrically connected to the flexible circuit board while connected to the connection part, and includes a plurality of electrode elements forming at least one positive electrode and at least one negative electrode; and a control part connected to the flexible circuit board so as to apply voltage to the light source element or the electrode elements.

### TECHNICAL FIELD

The present disclosure relates to a skin care device that considers convenience of a user and improves adhesion to skin.

### BACKGROUND ART

A device for skin care or treatment is implemented through various schemes. Skin receives microscopic stimulation and changes cell activity and the like, which may lead to skin improvement effects such as wrinkle improvement, lightening, and the like.

Schemes of stimulating the skin are largely divided into a non-invasive scheme of electrically stimulating the skin and an invasive scheme of physically stimulating the skin.

Examples of the non-invasive scheme may include a scheme of providing electrical stimulation in a high frequency band, an optical scheme of providing stimulation in a visible light band, a thermal scheme of providing stimulation by transferring heat energy, and the like.

In a conventional skin care device, it was common that one effect is provided by one device in terms of a care scheme, that is, a scheme of stimulating the skin. This is because when a plurality of functions are implemented by one device, mutual interference may occur and a total volume increases.

Further, a conventional portable skin care device that is in close contact with facial skin is poor in adhesion and in ease of use, so that a user prefers a hand-held device that, in a state of being held by a hand, needs to be directly in contact with a face or needs to be rubbed in a state of being in contact with the face. Therefore, it was difficult to perform other tasks while using the skin care device.

### DISCLOSURE

### Technical Problem

One purpose of the present disclosure is to provide a skin care device in which optical care and micro-current care are able to be performed at the same time without occurrence of electrical interference therebetween, usability is improved by including a structure that improves skin adhesion, and a patch that is in direct contact with skin is detachable and disposable to improve hygiene.

### Technical Solutions

According to one aspect of the present disclosure to achieve the above or another purpose, a skin care device characterized of including a flexible circuit board, at least one light source element disposed on one face of the flexible circuit board, a first casing for mounting the flexible circuit board therein, a hole defined in the first casing and exposing the light source element, a connector disposed on a face of the first casing including the hole defined therein, an electrode patch detachable from the connector, electrically connected to the flexible circuit board in a state of being coupled to the connector, and including a plurality of electrode elements forming at least one positive electrode and at least one negative electrode, and a controller connected to the flexible circuit board to apply a voltage to the light source element or the electrode element is provided.

Further, according to one aspect of the present disclosure, the skin care device characterized in that the flexible circuit board is formed to extend in different three directions, and each connector is disposed at each of both ends of the first casing corresponding to each of second and third direction extensions of the flexible circuit board is provided.

Further, according to one aspect of the present disclosure, the skin care device characterized in that the flexible circuit board is detachably connected to a hard circuit board having the controller disposed on a first direction extension thereof is provided.

Further, according to one aspect of the present disclosure, the skin care device characterized of further including a second casing for mounting the hard circuit board therein and connected to the first casing is provided.

Further, according to one aspect of the present disclosure, the skin care device characterized in that the first casing is partitioned by at least one first articulation for performing an articulation movement is provided.

Further, according to one aspect of the present disclosure, the skin care device characterized in that the first casing includes a plurality of heat dissipation plates between the other face of the flexible circuit board and the first casing is provided.

Further, according to one aspect of the present disclosure, the skin care device characterized in that the first casing further includes a second articulation linked with the second casing within a predefined angle range is provided.

Further, according to one aspect of the present disclosure, the skin care device characterized in that the first casing is made of elastic silicon and includes a cover of a transparent material inserted into the hole to shield the flexible circuit board from outside is provided.

Further, according to one aspect of the present disclosure, the skin care device characterized of further including a steel special use stainless (SUS) plate mounted in the first casing and stacked on the other face of the flexible circuit board is provided.

Further, according to one aspect of the present disclosure, the skin care device characterized in that the first casing includes a protrusion extending in a first direction and protruding around the flexible circuit board, and the second casing includes a container engaged with the protrusion at a portion thereof connected to the first casing to shield the portion thereof connected to the first casing from the outside is provided.

Further, according to one aspect of the present disclosure, the skin care device characterized in that the second casing includes a plurality of fixing wings respectively protruding toward second and third directional ends of the first casing to support a state of the first casing of being bent along a curved face of skin is provided.

Further, according to one aspect of the present disclosure, the skin care device characterized in that the electrode patch is formed by printing a conductive material on a plastic material by the electrode element, is in contact with skin, and stacks hydrogel capable of containing consumable drug thereon is provided.

Further, according to one aspect of the present disclosure, the skin care device characterized of further including the connector connected to the electrode patch on the second casing is provided.

### Advantageous Effects

Effects of a mobile terminal according to the present disclosure are as follows.

The present disclosure may provide convenience for the user to freely use their hands during skin care by improving adhesion to the skin in the skin care device.

The present disclosure simultaneously performs the optical care and the micro-current care and prevents the occurrence of the electrical interference therebetween to improve safety of a product.

The present disclosure is manufactured as a disposable product, which is a patch that is in close contact with the skin, that may be attached to and detached from the skin care device, thereby improving the user convenience and the hygiene.

The present disclosure is a waterproof and dustproof structure, which may improve stability of the product.

An additional range of applicability of the present disclosure will become apparent from a detailed description below. However, various changes and modifications within the spirit and range of the present disclosure may be clearly understood by those skilled in the art, and thus, the detailed description and specific embodiments such as preferred embodiments of the present disclosure should be understood to be given only as examples.

### DESCRIPTION OF DRAWINGS

FIG. 1 is a block diagram for illustrating a skin care device associated with the present disclosure.

FIG. 2 is an exploded perspective view of an embodiment of a skin care device associated with the present disclosure.

FIG. 3 is a combined perspective view in one direction of an embodiment of a skin care device associated with the present disclosure.

FIG. 4 is a combined perspective view in another direction of an embodiment of a skin care device associated with the present disclosure.

FIG. 5 is an exploded perspective view of another embodiment of a skin care device associated with the present disclosure.

FIG. 6 illustrates a combined view in one direction of another embodiment of a skin care device associated with the present disclosure.

FIG. 7 illustrates a combined view in another direction of another embodiment of a skin care device associated with the present disclosure.

FIG. 8 is a perspective view in one direction of an embodiment of a first casing associated with the present disclosure.

FIG. 9 is a perspective view in another direction of an embodiment of a first casing associated with the present disclosure.

FIG. 10 is an exploded view of an embodiment of a second casing associated with the present disclosure.

FIG. 11 is a perspective view of an embodiment of a second casing associated with the present disclosure.

FIG. 12 is a perspective view of an embodiment of a USB associated with the present disclosure.

### BEST MODE

Reference will now be made in detail to the preferred embodiments of the present invention, examples of which are illustrated in the accompanying drawings. In the drawings, the same or similar elements are denoted by the same reference numerals even though they are depicted in different drawings. The suffixes “module” and “unit”, which will be used hereinafter, are provided or used interchangeably in consideration of ease of description, and do not have distinguishable meaning or role. In the following description of the present invention, a detailed description of known functions and configurations incorporated herein will be omitted when it may make the subject matter of the present invention rather unclear. In addition, the accompanying drawings are provided only for better understanding of the present invention, and do not limit the technical spirit of the invention and include all changes, equivalents and substitutes within the scope and spirit of the invention.

Devices for skin care or treatment are implemented through various methods. When skin receives microstimulation, activity of cells, etc. are changed and thus skin improvement effects, such as wrinkle improvement, skin-whitening, etc. may be caused.

Methods of providing stimulation to the skin are divided into non-invasive methods in which electrical stimulation is provided to the skin, and invasive methods in which physical stimulation is provided to the skin.

As non-invasive methods, there are a high frequency method in which electrical stimulation of a high frequency band is provided to the skin, an optical method in which stimulation of visible light is provided to the skin, and a thermal method in which heat energy is transmitted so as to stimulate the skin.

In conventional skin care devices, one skin care device generally provides only one of effects respective care methods, i.e., skin stimulation methods.

Portable skin care devices are scarcely developed and demand therefor is low, and, if one skin care device implements a plurality of functions, interference thereamong may be caused and the overall volume of the skin care device may be increased.

Further, since most portable skin care devices are handheld devices which a user puts directly on his/her face or with which a user rubs his/her face, under the condition that the user grasps the device, it is difficult for the user to perform another operation simultaneously with use of the skin care device.

In order to solve such inconvenience, a method in which a face mask-type skin care device is worn by a user and is fixed to the back of the user head is proposed. However, such a method may cause large volume and heavy weight of the skin care device and is thus still not used simply.

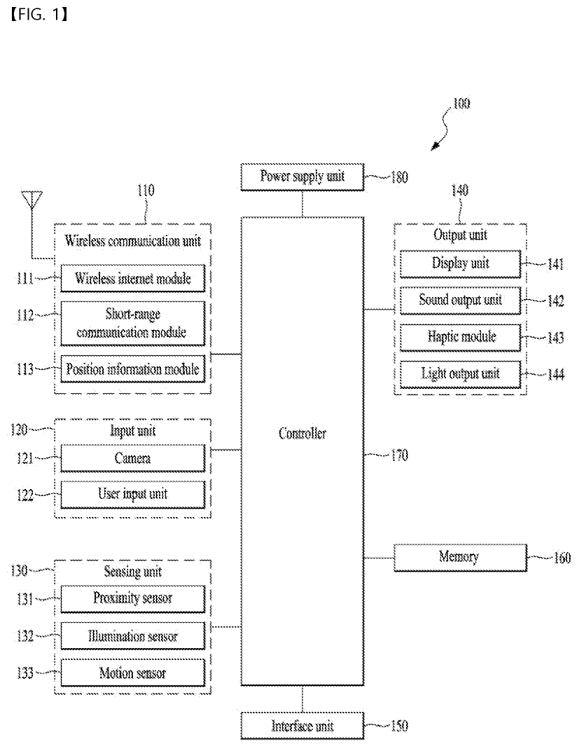

FIG. 1 is a block diagram illustrating a skin care device 100 in accordance with the present invention.

The skin care device 100 may include a wireless communication unit 110, an input unit 120, a sensing unit 130, an output unit 140, an interface unit 150, a memory 160, a controller 170, a power supply unit 180, etc.

The elements shown in FIG. 1 are not essential to implement the skin care device 100, and the skin care device 100 described herein may include a larger or smaller number of elements than the number of the above-described elements.

In more detail, the wireless communication unit 110 may include one or more modules facilitating wireless communication between the skin care device 100 and a wireless communication system, between the skin care device 100 and another external terminal, or between the skin care device 100 and an external server. Further, the wireless communication unit 110 may include one or more modules connecting the skin care device 100 to one or more networks.

The wireless communication unit 110 may include at least one of a wireless Internet module 111, a short-range communication module 112 and a position information module 113.

The short-range communication module 112 may support short-range communication using at least one of Bluetooth™, Radio Frequency Identification (RFID), Infrared Data Association (IrDA), Ultra Wideband, ZigBee, Near Field Communication (NFC), Wireless-Fidelity (Wi-Fi), Wi-Fi Direct, and Wireless Universal Serial Bus (USB) technologies. The short-range communication module 112 may support wireless communication between the skin care device 100 and a wireless communication system, between the skin care device 100 and another skin care device 100, or between the skin care device 100 and a network in which another skin care device 100 (or an external server) is located, through wireless area networks. The wireless area networks may be wireless personal area networks.

Here, another external terminal may be a mobile terminal or a wearable device (for example, a smart watch, smart glasses, a head mounted display (HMD), etc.), which may exchange data with (or interwork with) the skin care device 100 in accordance with the present invention. The short-range communication module 112 may sense (or recognize) a mobile terminal or a wearable device, which is communicable with the skin care device 100, around the skin care device 100. Further, if the sensed mobile terminal or wearable device is a device authenticated as communicating with the skin care device 100 in accordance with the present invention, the controller 170 may transmit at least a part of data processed by the skin care device 100 to the mobile terminal or the wearable device through the short-range communication module 112. Therefore, a user of the mobile terminal or the wearable device may use data, processed by the skin care device 100, through the mobile terminal or the wearable device. Otherwise, the skin care device 100 may receive data processed by the mobile terminal or the wearable device from the mobile terminal or the wearable device and thus perform a specific operation.

For example, the skin care device 100 may transmit data regarding a measured skin condition to the mobile terminal or the wearable device, such data may be stored in a database so that tendency of change in the skin condition is detected, and the skin care device 100 may receive feedback therefrom so that operation of the skin care device 100 is controlled.

Particularly, the skin care device 100 in accordance with the present invention may employ short-range communication technologies, such as Bluetooth™, Radio Frequency Identification (RFID), Infrared Data Association (IrDA), Ultra Wideband, ZigBee, Near Field Communication (NFC), and Wireless Universal Serial Bus (USB).

Thereamong, an NFC module provided in the skin care device 100 supports near field communication between terminals within a distance of about 10 cm. The NFC module may be operated in one of a card mode, a reader mode and a Peer-to-Peer (P2P) mode. In order to operate the NFC module in the card mode, the skin care device 100 may further include a security module which stores card information. Here, the security module may be a physical medium, such as a Universal Integrated Circuit Card (UICC) (for example, a Subscriber Identification Module (SIM) or a Universal SIM), a Secure micro SD and a sticker, or a logical medium embedded in the skin care device 100 (for example, an embedded SE (Secure Element)). Data exchange based on a Single Wire Protocol (SWP) may be carried out between the NFC module and the security module.

If the NFC module is operated in the card mode, the skin care device 100 may transmit card information stored therein to the outside, in the same manner as a traditional IC card.

If the NFC module is operated in the reader mode, the skin care device 100 may read data from an external tag. Here, data received from the tag by the skin care device 100 may be coded in NFC Data Exchange Format set by the NFC forum. The NFC forum regulates 4 Record Types. In more detail, 4 Record Type Definitions (RTDs), i.e., Smart Poster, Text, Uniform Resource Identifier (URI) and General Control, are regulated.

If the NFC module is operated in the Peer-to-Peer (P2P) mode, the skin care device 100 may perform P2P communication with another device. Here, Logical Link Control Protocol (LLCP) may be applied to P2P communication. For the purpose of P2P communication, connection between the skin care device 100 and another external terminal may be generated. Here, the generated connection may be divided into a connectionless mode which is terminated after exchange of one packet, and a connection-oriented mode in which packets are successively exchanged. Data and setup parameters for Bluetooth and Wi-Fi connection may be exchanged through P2P communication. However, since an NFC communication range is short, the P2P mode may be effectively used to exchange small amounts of data.

The position information module 113 serves to acquire the position (or the current position) of the skin care device 100 and, for example, may include a Global Positioning System (GPS) module or a Wi-Fi module. As one example, if the GPS module is used, the skin care device 100 may acquire the position of the skin care device 100 using signals transmitted from GPS satellites. As another example, if the Wi-Fi module is used, the skin care device 100 may acquire the position of the skin case device 100 based on information of a wireless Access Point (AP) receiving or transmitting a wireless signal with the Wi-Fi module. As circumstances demand, the position information module 113 may substitutively or additionally perform any function of another module of the wireless communication unit 110 so as to acquire data regarding the position of the skin care device 100. The position information module 113 is used to acquire the position (or the current position) of the skin care device 100, but the position information module 113 is not limited to a module which directly calculates or acquires the position of the skin care device 100.

The input unit 120 may include a camera 121 or an image input unit to receive an image signal and a user input unit 122 to receive information from a user (for example, a touch key, a mechanical key, etc.) Image data collected by the input unit 120 may be analyzed and processed into user control instructions.

The camera 121 processes an image frame, such as a still image or a moving picture acquired by an image sensor in a video call mode or in a scene mode. The processed image frame may be displayed by a display unit 141 or be stored in a memory 160.

The camera 121 includes at least one of a camera sensor (for example, a CCD, a CMOS, etc.), a photo sensor (or an image sensor) and a laser sensor.

The camera 121 and the laser sensor may be combined with each other and thus sense touch of an object to be sensed in a 3D image. The photo sensor may be stacked on a display device, and such a photo sensor scans movement of the object to be sensed, which is brought close to a touchscreen. In more detail, the photo sensor includes photo diodes and transistors (TRs) in rows and columns and thus scans the object to be sensed, placed on the photo sensor, using an electrical signal changed according to an amount of light applied to the photo diodes. That is, the photo sensor may calculate coordinates of the object to be sensed according to change in the amount of light and thereby position information of the object to be sensed may be acquired.

The camera 121 provided in the skin care device 100 may perform functions of photographing the condition of a skin surface, to which the skin care device 100 is attached. If the skin care device 100 is provided with the display unit 141, the photographed skin surface condition may be output through the display unit 141 so that a user may confirm the same.

The user input unit 122 serves to receive information from a user and, when information is input to the skin care device 100 through the user input unit 122, the controller 170 may control operation of the skin care device 100 so as to correspond to the input information. The user input unit 122 may include a mechanical input means (or a mechanical key, for example, a button, a dome switch, a jog wheel or a jog switch located on the front, rear or side surface of the skin care device 100), and a touch-type input means. For example, the touch-type input means may include a virtual key displayed on a touchscreen through processing by software, a soft key or a visual key, or include a touch key disposed in other regions than the touchscreen. The virtual key or the visual key having various shapes may be displayed on the touchscreen. For example, the virtual key or the visual key may include a graphic, a text, an icon, a video or a combination thereof.

The sensing unit 130 may include one or more sensors to sense at least one of information within the skin care device 100, information regarding surrounding environment of the skin care device 100 and user information. For example, the sensing unit 130 may include at least one of a proximity sensor 131, an illumination sensor 132, a touch sensor, an acceleration sensor, a magnetic sensor, a G-sensor, a gyroscope sensor, a motion sensor 133, an RGB sensor, an infrared sensor (IR sensor), a finger scan sensor, an ultrasonic sensor, an optical sensor (for example, a camera), a microphone, a battery gauge, environment sensors (for example, a barometer, a hygrometer, a thermometer, a radioactivity sensor, a heat sensor, a gas sensor, etc.), and chemical sensors (for example, an electronic nose, a health care sensor, a biometric sensor, etc.). The skin care device 100 in accordance with the present invention may use a combination of information sensed by at least two of the above-described sensors.

The sensing unit 130 senses at least one of information within the skin care device 100, information regarding surrounding environment of the skin care device 100 and user information, and generates a sensing signal corresponding to the sensed information. The controller 170 may control driving or operation of the skin care device 100 or perform data processing, functions or operations regarding application programs installed in the skin care device 100, based on the sensing signal.

The proximity sensor 131 detects an object approaching a designated detection surface or whether or not an object is present around the proximity sensor 131 using electromagnetic force or infrared light without mechanical contact. The proximity sensor 131 may be disposed in the inner area of the skin care device 100 surrounded by a touchscreen, which will be described later, or disposed around the touchscreen.

For example, the proximity sensor 131 may be a transmissive photoelectric sensor, a direct reflective photoelectric sensor, a mirror reflective photoelectric sensor, a high-frequency oscillating proximity sensor, a capacitive proximity sensor, a magnetic proximity sensor, an infrared proximity sensor, etc. If the touchscreen is a capacitive type, the proximity sensor 131 may be configured to detect proximity of an object having conductivity through change in an electric field according to proximity of the object. In this case, the touchscreen (or a touch sensor) itself may be regarded as a proximity sensor.

Hereinafter, for convenience of description, an action in which an object is brought close to the touchscreen without contact and thus it is recognized that the object is located on the touchscreen is referred to as “proximity touch”, and an action in which an object actually contacts the touchscreen is referred to as “contact touch”. A proximity touch position of an object on the touchscreen means a position of the touchscreen vertically corresponding to the object when the object is in the proximity touch state on the touchscreen. The proximity sensor 131 may sense proximity touch and a proximity touch pattern (for example, a proximity touch distance, a proximity touch direction, a proximity touch speed, a proximity touch time, a proximity touch position, a proximity touch moving state, etc.). The controller 170 may process data (or information) corresponding to the proximity touch operation and the proximity touch pattern sensed by the proximity sensor 131 and output visual information corresponding to the processed data on the touchscreen. Further, the controller 170 may control the skin care device 100 so as to process different operations or data (or information) according to whether or not touch of the object at the same point of the touchscreen is proximity touch or contact touch.

The touch sensor senses touch (or touch input) applied to the touchscreen (or the display unit 141) using at least one of various touch types, i.e., a resistive type, a capacitive type, an infrared type, an ultrasonic type, a magnetic field type, etc.

For example, the touch sensor may be configured to convert change in pressure applied to a specific region of the touchscreen or capacitance generated from a specific region of the touchscreen into an electrical input signal. The touch sensor may be configured to detect a touch position of an object on the touchscreen, a touch area of the object, a touch pressure of the object, a capacitance in touch of the object, etc. Here, the object is an article touching the touchscreen and, for example, may be a finger, a touch pen or stylus, or a pointer.

The controller 170 may perform different control according to kinds of objects touching the touchscreen (or a touch key provided in other regions than the touchscreen), or perform equal control regardless of kinds of objects touching the touchscreen. Whether or not different control is performed or equal control is performed according to kinds of objects may be determined according to the current operating state of the skin care device 100 or an application program which is being executed.

The above-described touch sensor or proximity sensor may independently used or be combined to sense various types of touch, such as short (or tap) touch, long touch, multi-touch, drag touch, flick touch, pinch-in touch, pinch-out touch, swipe touch, hovering touch, etc., on the touchscreen.

The ultrasonic sensor may recognize position information of an object to be sensed, using ultrasonic waves. The controller 170 may calculate the position of a wave source through information sensed by the optical sensor and a plurality of ultrasonic sensors. The position of the wave source may be calculated using the principle that light is much faster than ultrasonic waves, i.e., that an arrival time of light to the optical sensor precedes an arrival time of ultrasonic waves to the ultrasonic sensor. In more detail, the position of the wave source may be calculated using a time difference between the light arrival time and the ultrasonic wave arrival time.

If the touch sensor senses touch input, a signal (or signals) corresponding thereto is transmitted to a touch controller. The touch controller processes the signal(s) and transmits corresponding data to the controller 170. Thereby, the controller 170 recognizes which area of the display unit 141 is touched, etc. Here, the touch controller may be an element provided separately from the controller 170, or may be the controller 170 itself.

The output unit 140 generates output regarding sight, hearing or tactile sense, and may include at least one of the display unit 141, a sound output unit 142, a haptic module 143, and a light output unit 144. The display unit 141 may form a layered structure with the touch sensor or be formed integrally with the touch sensor, thus forming the touchscreen. Such a touchscreen may function as the user input unit 122 providing an input interface between the skin care device 100 and a user and provide an output interface between the skin care device 100 and the user.

The display unit 141 displays (outputs) information processed by the skin care device 100. For example, the display unit 141 may display execution picture information of application programs operated in the skin care device 100, or user interface (UI) or graphical user interface (GUI) information according to the execution picture information.

The interface unit 150 serves as an interface with various kinds of external devices connected to the skin care device 100. The interface unit 150 may include at least one of wired/wireless headset ports, an external charger port, wired/wireless data ports, a memory card port, and a port for connection with a device provided with an identification module. In response to connection of an external device to the interface unit 150, the skin care device 100 may perform proper control regarding the connected external device.

The memory 160 stores data supporting various functions of the skin care device 100. The memory 160 may store a plurality of application programs (or applications) executed by the skin care device 100 and data and commands for operating the skin care device 100. At least some of the application programs may be downloaded from an external server through wireless communication. Further, at least some of the application programs may be pre-loaded in the skin care device 100 at the factory so as to perform basic functions. The application programs are stored in the memory 160 and installed in the skin care device 100, and may thus be executed by the controller 170 so as to perform operation (or functions) of the skin care device 100.

The controller 170 controls the overall operation of the skin care device 100 in addition to operations regarding the application programs. The controller 170 may process signals, data, information, etc. input or output through the above-described elements or execute the application programs stored in the memory 160, thereby providing proper information or functions to a user or processing the information.

In order to execute the application program stored in the memory 160, the controller 170 may control at least some of the elements described with reference to FIG. 1. Further, in order to execute the application program, the controller 170 may operate at least two of the elements of the skin care device 100 in combination with each other.

The power supply unit 180 receives external power or internal power and then supplies power to the respective elements of the skin care device 100 under the control of the controller 170. The power supply unit 180 includes a battery, and the battery may be an internal battery or a replaceable battery.

At least some of the respective elements may be operated in cooperation with each other so as to operate or control the skin care device 100 or to implement a control method of the skin care device 100. Further, the operation, control and control method of the skin care device 100 may be implemented in the skin care device 100 by executing at least one application program stored in the memory 160.

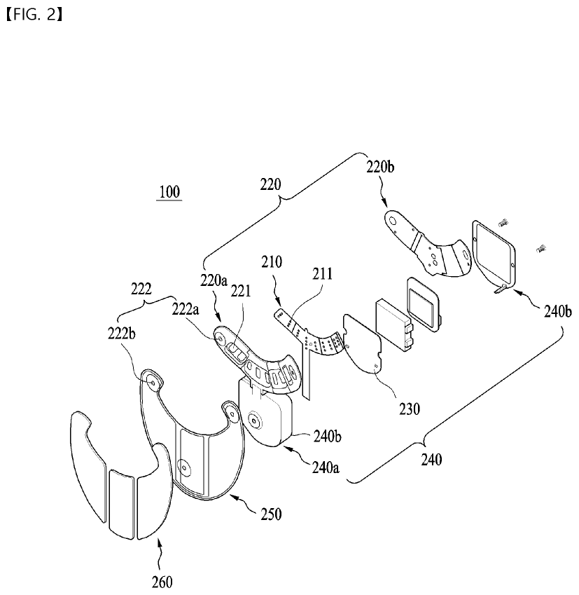

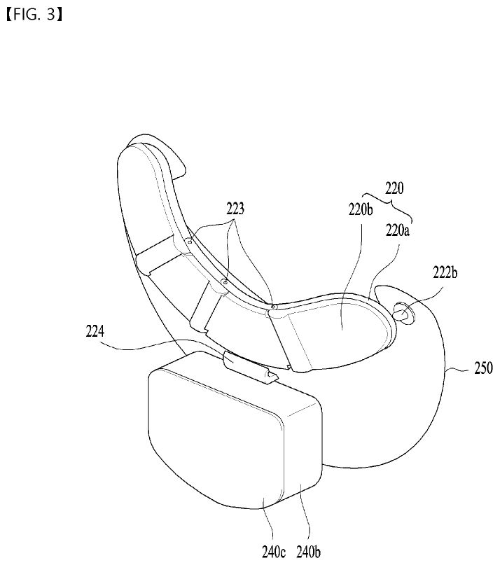

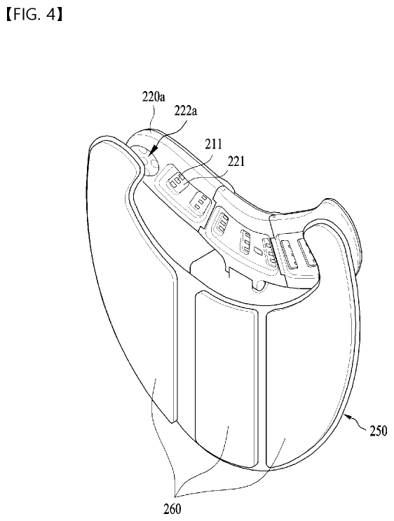

FIG. 2 is an exploded perspective view of an embodiment of the skin care device 100 associated with the present disclosure. FIGS. 3 and 4 are combined perspective views in different directions illustrating an embodiment of the skin care device 100 associated with the present disclosure.

In order to solve the above-mentioned conventional problems, the present disclosure provides the skin care device 100 in which optical energy and electric energy are output simultaneously or selectively, but a light source element and an electrode element do not electrically interfere with each other, and a micro-current care portion (an electrode patch 250) attached to the skin is manufactured as a disposable product that may be attached to and detached from the device.

A flexible circuit substrate 210, which is made of a non-conductive material, may mount at least some of electronic parts thereon.

The flexible circuit board 210 may include at least one light source element 211 on one face thereof.

The light source element 211 may stimulate and treat the skin by irradiating the skin through light-emitting.

The skin care device 100 may include the electrode element (not shown) for stimulating and treating the skin by stimulating the skin through a micro-current.

The electrode element may be disposed on the flexible circuit board 210. However, in this case, it may be accompanied by a problem of electrical interference with the light source element 211 disposed on the flexible circuit board 210.

Therefore, it is advantageous that the electrode element is disposed on another board to prevent the electrical interference with the light source element 211 disposed on the flexible circuit board 210.

When the light source element 211 and the electrode element are respectively arranged on different boards, a structure of receiving a voltage from the controller 170 is an important factor.

The flexible circuit board 210 including the light source element 211 may be mounted in a non-conductive first casing 220, and the electrode element may be disposed on the electrode patch 250 connected to the first casing 220.

The controller 170 may be connected to the flexible circuit board 210 and applies the voltage to the light source element 211, and may apply the voltage to the electrode element through a connector 222 for connecting the first casing and the electrode patch 250 with each other.

The controller 170 may simultaneously or selectively apply the voltage to the light source element 211 and the electrode element to stimulate and treat the skin.

When a temperature sensor is disposed in the skin care device 100, a skin temperature may be fed back to control the voltage application by the controller 170.

Hereinafter, for convenience of description, a side in contact with the skin is defined as an inner side and a corresponding opposite side is defined as an outer side.

The light source element 211 may be exposed to the inner side by a light-pass hole 221 defined in the first casing 220 to irradiate optical energy to the skin.

The first casing 220 may define therein an accommodation space in which the flexible circuit board 210 is mounted by coupling a first casing inner plate 220a that forms an inner casing with a first casing outer plate 220b that forms an outer casing. Further, the first casing 220 may include the light-pass hole 221 defined in the first casing inner plate 220a.

The electrode patch 250 may be connected to the electrode element in a scheme of printing a conductive material such as silver (Ag), carbon (C), or the like on a plastic material to reduce a thickness of the electrode patch 250.

The conductive material disposed on the electrode patch 250 may be connected to the connector 222 of a metal material to be connected to the controller along the conductive material formed on the flexible circuit board 210.

The connector 222 may include a plurality of connectors, and electrode elements respectively connected to the plurality of connector 222 may form at least one positive electrode and at least one negative electrode.

The electrode patch 250 may be disposed on an inner face of the first casing 220 to be directly in contact with or be close to the skin, thereby allowing the micro-current to flow inside the skin.

The controller 170 may measure an electrical resistance of the skin to adjust the voltage, thereby allowing the current to flow constantly without changing.

The electrode patch 250 is preferably disposed outside a region where the optical energy is irradiated from the light-pass hole 221 to the skin.

The electrode patch 250 may be manufactured in various shapes suitable for a size and a shape of the skin detachably connected to and in contact with the first casing 220.

The connector 222 may be manufactured to selectively detach the electrode patch 250 and the first casing 220 from each other and electrically connect the electrode patch 250 and the first casing 220 to each other such that the voltage is applied from the controller 170 to the electrode element.

The connector 222 may be a snap button made of a metal material in one embodiment.

The electrode patch 250 may be formed by stacking hydrogel 260 in contact with the skin.

The hydrogel 260, which is also referred to as hydrated gel, refers to a material that may not dissolve in an aqueous environment and may contain a considerable amount of water as a network structure in which a water-soluble polymer forms a three-dimensional crosslink by physical or chemical bonding.

The hydrogel 260 may generate skin adhesion, and an easily adjustable porous structure of the hydrogel 260 may enable drug loading on the gel, so that a specific drug concentration on the skin may be maintained continuously over a long period of time.

A structure of the electrode patch 250 that may be detached from the first casing 220 may be conveniently replaced when a different specific drug is loaded on the hydrogel 260 or when a specific drug is exhausted.

As described above, the light-source device 211 and the electrode element may be separated from each other to avoid the electrical interference therebetween, and the electrode patch 250 to which the hydrogel 260 is attached may be manufactured as the disposable product and may be used by being connected to the device by the user, thereby improving the ease of use and the hygiene.

When the electrode patch 250 is manufactured as the disposable product, a structure of the device connected to the electrode patch 250 is an important factor to improve a shape freedom of the electrode patch 250, thereby improving the skin adhesion.

The flexible circuit board 210 may be formed to extend in different three directions, and the first casing 220 may be formed to mount the flexible circuit board 210 extending in two directions and three directions therein.

The controller 170 may be connected to a first direction extension of the flexible circuit board 210, and may be mounted in the second casing 240, which is connected to the first casing 220.

The first substrate 220 extending in both directions may be flexible, and may be bent to fit a curved face of the skin to improve the skin adhesion.

It is preferable that the plurality of connectors 222 are respectively arranged at both ends of the first casing 220 in which second and third direction extensions of the flexible circuit board are mounted.

However, a structure supported at three points is more stable. Therefore, in order to fix the second casing 240 connected to the first casing 220, the connector 222 may be further disposed in the second casing 240.

The controller 170 may be formed on a hard circuit substrate 230 connected to the flexible circuit board 210 to improve device stability.

The hard circuit substrate 230 may be detachably connected to the flexible circuit board 210 to be easily assembled and easily replaced when the hard circuit substrate 230 is defective, and may reduce a size of the device to reduce a weight of the device.

In the second casing 240, a second casing cover 240c for covering the accommodation space in which the hard circuit substrate 230 is accommodated is fastened using a bolt and the like, so that after replacement of parts replacement and repair may be facilitated after the second casing cover 240c is removed.

Hereinafter, an embodiment in which the first casing 220 has an articulation structure in order to have flexibility will be described in detail with reference to FIGS. 2 to 4.

The first casing 220 may be partitioned by at least one first articulation 223 performing an articulation movement.

An angle of the first casing 210 may be adjusted to fit the face by bending the first articulation 223.

In order to prevent the flexible circuit board 210 mounted inside the first casing 220 from being excessively bent and damaged, the first articulation 223 may be formed to perform the articulation movement within a predefined angle range.

The first casing 220 may be connected to the second casing 240 through a second articulation 224 that performs an articulation movement within a predefined angle range.

The second articulation 224 may perform the articulation movement within the predefined angle range to prevent the first casing 220 from being excessively bent or prevent a connection between the flexible circuit board 210 and the hard circuit substrate 230 from being damaged.

In order to prevent heat generated from the light source element 211 from increasing in a local area, the first casing 220 may dispose a copper plate (heat dissipation plate) between the first casing outer plate 220b that forms the outer casing of the first casing 220 and the flexible circuit board 210 to dissipate the heat generated from the light source element 211 over a large area.

It is preferable that the copper plate (heat dissipation plate) is disposed in a region partitioned by the first articulation 223 such that the articulation of the first articulation 223 is not disturbed.

When the first casing 220 is formed to have the articulation structure, dust and moisture may penetrate through the articulation. Accordingly, the first casing 220 may surround the flexible circuit board 210 so as to shield the flexible circuit board 210 from the outside, thereby providing the skin care device 100 that is waterproof and dustproof.

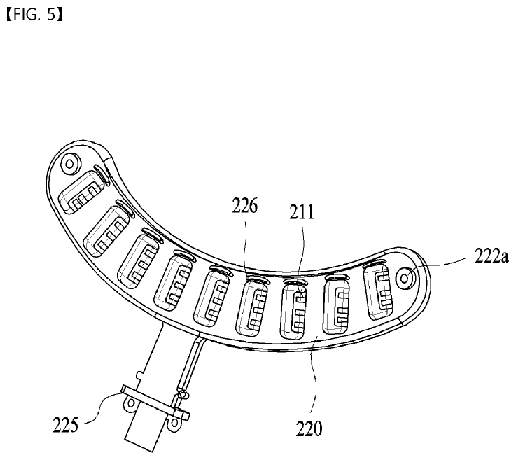

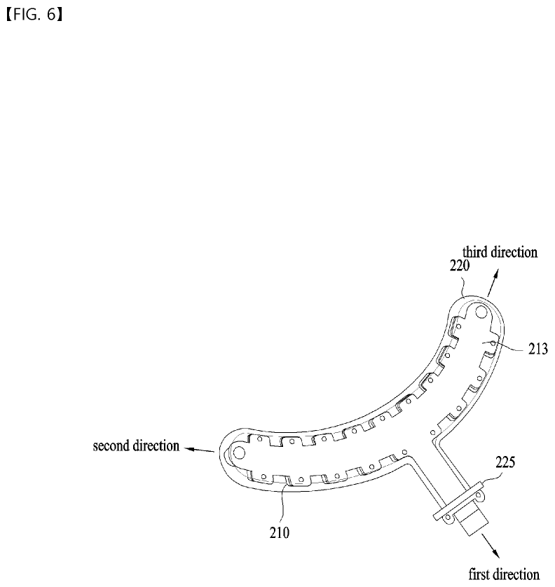

FIGS. 5 and 6 illustrate an embodiment in which the first casing 210 is made of silicon to shield the flexible circuit board 210 from the outside.

The first casing 220 is integrally formed and made of the silicon material, and includes a cover 226 made of a transparent material inserted into a light-pass hole 221 to shield the flexible circuit board 210 from the outside.

The cover 226 of the transparent material may allow light emitted from the light-source device 211 to be irradiated to a skin surface and may fix a contact between the light-source device 211 and the conductive material printed on the flexible circuit board 210.

The embodiment may include a SUS plate 213 that is mounted in the first casing 220 and that is deposited on an outer face of the flexible circuit board 210.

Unlike the first casing 220 formed to have the articulation structure, in the embodiment, the SUS plate 213 may be continuously formed along the flexible circuit board 210.

The SUS plate 213 may prevent the heat generated from the light source element 211 from being concentrated in the local area and may control the shape of the flexible circuit board 210.

The first casing 220 may include a protrusion 225 protruding around the flexible circuit board 210 that extends in the first direction to be connected to the second casing 240.

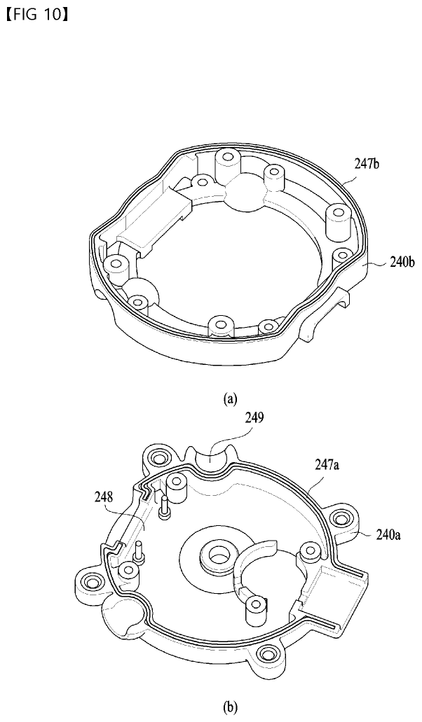

Referring to FIG. 10, the second casing 240 may include a container 248 engaged with the protrusion to fix the first casing 220 and prevent the dust and the moisture from flowing to a connection between the first and second casings.

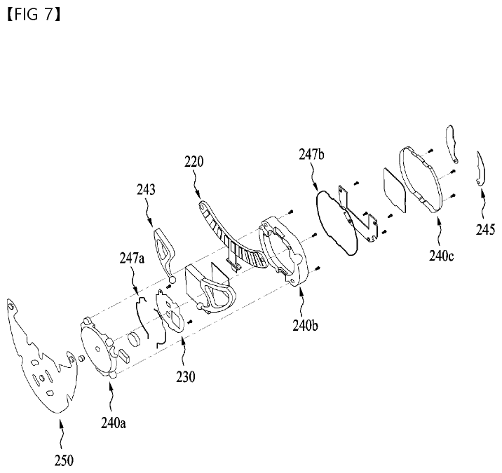

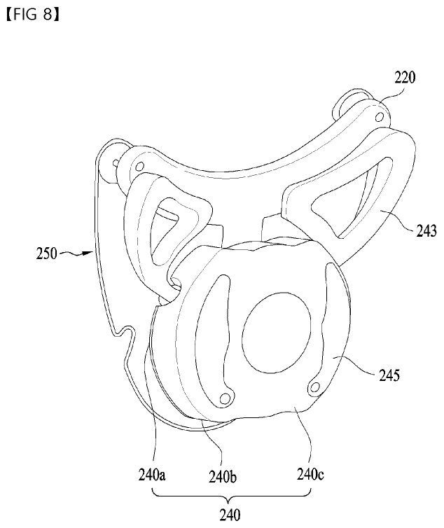

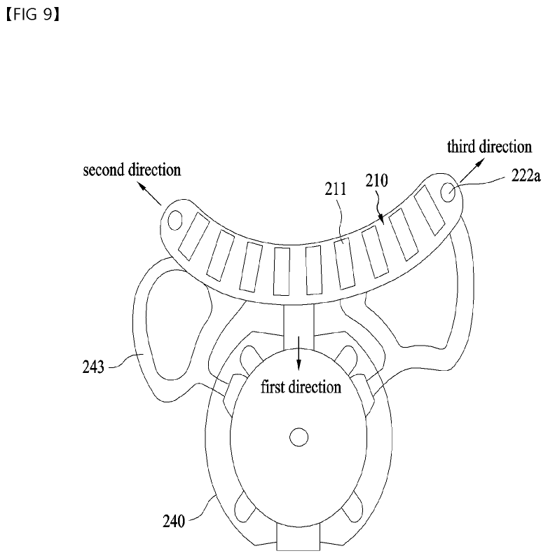

The first casing 220 made of the silicon material may include a position fixing wing 243 to maintain a shape thereof. FIGS. 7 to 9 illustrate an exploded view and combined views of the skin care device including the position fixing wing 243.

The second casing 240 may include the position fixing wing 243 that protrudes toward an end of the first casing 220 to support a state of the first casing 220 of being bent along the curved face of the skin.

The position fixing wing 243 may include a plurality of position fixing wings to respectively protrude toward a second directional end and a third directional end of the first casing 220 and respectively support the both ends.

The position fixing wing 243 is preferably connected to the second casing 240 in a linkable manner to be moved within a predefined angle range.

The second casing 240 may be formed by coupling a second casing inner plate 240a that forms an inner face of the second casing 240, a second casing body 240b that defines a space in which the hard circuit substrate 230 is accommodated, and a second casing cover 240c that covers the accommodation space with each other.

Hereinafter, a coupling structure of the second casing 240 in which the position fixing wing 243 is connected to the second casing 240 in the linkable manner and which is for waterproof and dustproof purposes will be described.

FIG. 10 is perspective views of the second casing inner plate 240a and the second casing body 240b.

An end of the position fixing wing 243 may have a spherical shape and may be inserted into a groove 249 defined between the second casing inner plate 240a and the second casing body 240b engaged with each other to be linked with the second casing inner plate 240a and the second casing body 240b.

Rubber packings 247a and 247b of an elastic material are inserted into a connection of the second casing inner plate 240a and the second casing body 240b and a connection of the second casing body 240b and the second casing cover 240c to prevent water from seeping inside.

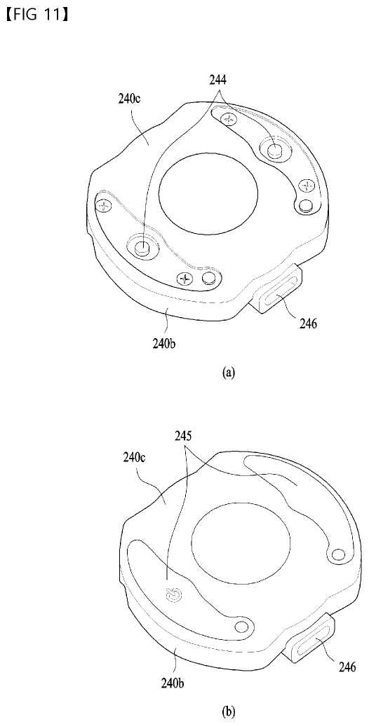

FIG. 11 illustrates a perspective view ((a) in FIG. 11) of the second casing cover 240c having a manipulation unit 244 such as a power source and the like, and a perspective view ((b) in FIG. 11) of the second casing cover 240c where the manipulation unit 244 is covered with a manipulation button cover 245.

The manipulation unit 244 of the skin care device 100 disposed on the second casing cover 240c may be formed in a tact type to prevent the water from entering a gap. Further, the manipulation unit cover 245 may cover a top of the manipulation unit 244 to vicinity of the gap to block a entering path of the water from outside.

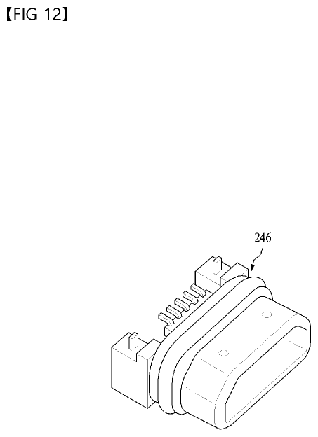

FIG. 12 is a perspective view illustrating a waterproof charging terminal 246.

The skin care device 100 may mount a battery therein as a power supply. In addition, the battery may be disposed in a form capable of being recharged. One example is a lithium-ion battery.

The rechargeable battery may be connected to the charging terminal 246 to be charged wiredly.

The charging terminal 246 is a waterproof USB that may prevent the water from seeping inside the second casing 240 through the charging terminal 246.

Although not shown, the skin care device 100 of the present disclosure may treat the skin in a scheme of transmitting vibration energy in addition to the optical scheme or the thermal scheme.

The skin care device 100 of the present disclosure may include the haptic module described above to implement the scheme of transmitting the vibration energy. As one example of the haptic module, a vibration motor may be disposed. The vibration energy may be especially transmitted in a form of 50 to 60 Hz.

It is apparent to those skilled in the art that the present disclosure may be embodied in other specific forms within a range that does not depart from the spirit and essential features of the present disclosure.

The above detailed description should not be construed as limiting in all respects, but should be considered illustrative. The range of the present disclosure should be determined by rational interpretation of the appended claims, and all changes within the equivalent range of the present disclosure are included in the range of the present disclosure.

1. A skin care device comprising:
a flexible circuit board;
at least one light source element disposed on one face of the flexible circuit board;
a first casing for mounting the flexible circuit board therein;
a hole defined in the first casing and exposing the light source element;
a connector disposed on a face of the first casing including the hole defined therein;
an electrode patch detachable from the connector, electrically connected to the flexible circuit board in a state of being coupled to the connector, and including a plurality of electrode elements forming at least one positive electrode and at least one negative electrode; and
a controller connected to the flexible circuit board to apply a voltage to the light source element or the electrode element.

2. The skin care device of claim 1, wherein the flexible circuit board is formed to extend in different three directions, and
wherein each connector is disposed at each of both ends of the first casing corresponding to each of second and third direction extensions of the flexible circuit board.

3. The skin care device of claim 2, wherein the flexible circuit board is detachably connected to a hard circuit board having the controller disposed on a first direction extension thereof.

4. The skin care device of claim 3, wherein the skin care device characterized of further including a second casing for mounting the hard circuit board therein and connected to the first casing is provided.

5. The skin care device of claim 4, wherein the first casing is partitioned by at least one first articulation for performing an articulation movement.

6. The skin care device of claim 5, wherein the first casing includes a plurality of heat dissipation plates between the other face of the flexible circuit board and the first casing.

7. The skin care device of claim 5, wherein the first casing further includes a second articulation linked with a second casing within a predefined angle range.

8. The skin care device of claim 4, wherein the first casing is made of elastic silicon and includes a cover of a transparent material inserted into the hole to shield the flexible circuit board from outside.

9. The skin care device of claim 8, further comprising:
a steel special use stainless (SUS) plate mounted in the first casing and stacked on the other face of the flexible circuit board.

10. The skin care device of claim 8, wherein the first casing includes a protrusion extending in a first direction and protruding around the flexible circuit board, and
wherein a second casing includes a container engaged with the protrusion at a portion thereof connected to the first casing to shield the portion thereof connected to the first casing from the outside.

11. The skin care device of claim 8, wherein a second casing includes a plurality of fixing wings respectively protruding toward second and third directional ends of the first casing to support a state of the first casing of being bent along a curved face of skin.

12. The skin care device of claim 1, wherein the electrode patch is formed by printing a conductive material on a plastic material by the electrode element, is in contact with skin, and stacks hydrogel capable of containing consumable drug thereon.

13. The skin care device of claim 4, further comprising:
the connector connected to the electrode patch on a second casing.
